(19) United States
(12) Patent Application Publication
(10) Pub. No.: US 2008/0199421 A1
(43) Pub. Date: Aug. 21, 2008

(54) W/O EMULSION

(75) Inventor: Raluca LORANT, Thiais (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/031,879

(22) Filed: Feb. 15, 2008

Related U.S. Application Data
(60) Provisional application No. 60/892,268, filed on Mar. 1, 2007.

(30) Foreign Application Priority Data
Feb. 21, 2007 (FR) 07 53392

(51) Int. Cl. A61K 31/74; A61P 17/00

(52) U.S. Cl. 424/78.03

(57) ABSTRACT

The invention relates to a composition preferably for topical application in the form of a water-in-oil emulsion containing at least one emulsifying silicone elastomer and at least one semi-crystalline polymer. The invention also relates to the use of the composition according to the invention for skincare, and in particular for nourishing the skin, preventing transepidermal water loss and protecting the skin.

### REFERENCE TO PRIOR APPLICATIONS

This application claims priority to U.S. provisional application 60/892,268 filed Mar. 1, 2007, and to French patent application 0753392 filed Feb. 21, 2007, both incorporated herein by reference.

### FIELD OF THE INVENTION

The invention relates in a preferred embodiment to a composition in the form of a water-in-oil (W/O) emulsion comprising an aqueous phase, an oxyalkylenated or glycerylated elastomeric organopolysiloxane, and a semi-crystalline polymer. The invention also relates to the uses of this composition, in particular as a skincare product.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In the cosmetics field, emulsions of water-in-oil (W/O) type are recognized as being effective for skincare due to the fact that they comprise an oily continuous phase and that they therefore make it possible to form, at the surface of the skin, a lipid film which prevents transepidermal water loss and protects the skin against outside attacks. These emulsions are particularly suitable for protecting and nourishing the skin, and in particular for treating dry skin. However, W/O emulsions are often criticized for being too greasy and tacky and for having a fragile stability. Generally, they do not provide freshness and they are often too rich in oils to be used during the summer or in hot countries.

In order to overcome these drawbacks, it has been proposed to prepare W/O emulsions with a high content of aqueous phase and of water. Thus, document EP-A-1,068,851 describes the use of an oxyalkylenated elastomeric organopolysiloxane as emulsifier for stabilizing W/O emulsions containing at least 70% of aqueous phase. However, the compositions obtained according to this document, although they effectively provide a certain freshness upon application due to the very high amount of aqueous phase, do not provide the known benefits of W/O emulsions, such as the nutrition of dry skin, due to the volatile nature of the oils that they contain. This is because, volatile oils, by definition, evaporate and, while they provide a pleasant feeling of freshness when spread, they do not provide any treating effect since they do not remain on the skin. Furthermore, the chemical nature of the silicone emulsifier imposes limits within the nature of the oils making up the oily phase: for reasons of feasibility and stability, it should contain predominantly silicone oils and/or volatile oils which should represent at least 15% by weight of the total weight of the composition. In addition, the emulsions described in this document require the presence of an electrolyte in the aqueous phase in order to ensure an acceptable level of stability. Now, some skin, particularly reactive skin, may poorly tolerate compositions containing an amount of electrolytes such as those described in the examples of this document (2.5%).

Moreover, document EP-A-1,136,058 also proposes W/O emulsions providing freshness upon application to the skin, by virtue of the presence of a high amount of aqueous phase. However, as in the document previously cited, these compositions stabilized by an oxyalkylenated elastomeric organopolysiloxane pose the same problems, i.e. the presence of volatile, in particular silicone, oils in a predominant proportion.

### SUMMARY OF THE INVENTION

There remains therefore the need for a composition in the form of a W/O emulsion which does not have the drawbacks of the prior art and in particular which provides:
an effect of freshness on application, in particular by virtue of the presence of a high degree of aqueous phase,
a nutritional effect, which is the primary benefit of emulsions with a continuous oily phase,
   while at the same time showing good stability over time.

The inventor has now found, surprisingly, that it is possible to obtain a composition that has the desired advantages and can contain non-volatile oils, using a water-in-oil emulsion containing an emulsifying silicone elastomer and a specific lipophilic polymer. The term “emulsifying silicone elastomer” is intended to mean a silicone elastomer comprising at least one hydrophilic chain, which confers emulsifying properties on it.

### DETAILED DESCRIPTION OF THE PREFERRED EMBODIMENTS

One subject of the invention is a composition, preferably suitable for topical application, in the form of a water-in-oil emulsion comprising an aqueous phase dispersed in an oily phase, and comprising at least one emulsifying silicone elastomer and at least one semi-crystalline polymer.

Since the composition is preferably for topical application, it contains a physiologically acceptable medium. The term “physiologically acceptable medium” is intended to mean a nontoxic medium compatible with the skin (including the inside of the eyelids), the mucous membranes, the hair or the lips of human beings. Since the composition preferably constitutes a cosmetic composition, it has an agreeable appearance, odour and feel.

The composition of the invention has the advantage of giving an effect of freshness when applied to the skin, while at the same time having a good nutritional effectiveness on the skin and at the same time showing good stability over time. In addition, it may contain a substantial amount of non-volatile oils without being unstable.

### Emulsifying Silicone Elastomer

The term “silicone elastomer” is intended to mean a partially or completely crosslinked organopolysiloxane, which is a flexible and deformable material having viscoelastic properties. Its modulus of elasticity is such that this material withstands deformation and has a limited capacity for extension and contraction. This material is capable of returning to its original shape following stretching.

The emulsifying silicone elastomer used according to the invention is a crosslinked elastomeric organopolysiloxane comprising at least one hydrophilic chain, it being possible for this chain to be in particular oxyalkylenated or glycerylated. The emulsifying silicone elastomer can therefore be chosen from silicone elastomers comprising at least one oxyalkylenated chain and/or one glycerylated chain.

The silicone elastomer comprising at least one oxyalkylenated chain can be obtained in particular by addition reaction and crosslinking of a diorganopolysiloxane containing at least two hydrogens each linked to a silicon (A1), and of a polyoxyalkylene having at least two ethylenically unsaturated groups (B1), in particular in the presence of a catalyst (C1), in particular a platinum catalyst, as described, for example, in documents U.S. Pat. No. 5,236,986 and U.S. Pat. No. 5,412,004.

Compound (A1) is the base compound for the formation of elastomeric organopolysiloxane, and the crosslinking takes place via an addition reaction of compound (A1) with compound (B1) in the presence of catalyst (C1).

Compound (B1) is advantageously an oxyethylenated and/or oxypropylenated compound containing at least two vinyl groups in the α-ω position of the silicone chain, which will react with Si—H bonds of compound (A1). Compound (B1) may in particular be a polyoxyalkylene (in particular polyoxyethylene and/or polyoxypropylene) containing dimethylvinylsiloxy end groups.

The organic groups linked to the silicon atoms of compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Compound (A1) may thus be chosen from methylhydrogenopolysiloxanes containing trimethylsiloxy end groups, dimethylsiloxane-methylhydrogenosiloxane copolymers containing trimethylsiloxy end groups, cyclic dimethylsiloxane-methylhydrogenosiloxane copolymers and di-methylsiloxane-methylhydrogenosiloxanelaurylmethyl-siloxane copolymers containing trimethylsiloxy end groups.

Compound (C1) is the crosslinking reaction catalyst, and is in particular chosen from chloroplatinic acid, chloroplatinic acid-olefin complexes, chloroplatinic acid-alkenylsiloxane complexes, chloroplatinic acid-diketone complexes, platinum black and platinum on a support.

The catalyst (C1) is preferably added in an amount of from 0.1 to 1000 parts by weight, better still from 1 to 100 parts by weight, as clean platinum metal, per 1000 parts by weight of the total amount of compounds (A1) and (B1).

In particular, the silicone elastomer comprising at least one oxyalkylenated chain can be obtained by reaction of polyoxyalkylene (in particular polyoxyethylene and/or polyoxypropylene) containing dimethylvinylsiloxy end groups and of methylhydrogenopolysiloxane containing trimethylsiloxy end groups, in the presence of platinum catalyst.

The silicone elastomer comprising at least one oxyalkylenated chain, used according to the invention, is preferably a silicone elastomer comprising at least one oxyethylenated chain.

In addition, the silicone elastomer comprising at least one oxyalkylenated chain is preferably carried in the form of a gel in at least one hydrocarbon-based oil and/or one silicone oil. In these gels, the elastomer comprising at least one oxyalkylenated chain is commonly in the form of nonspherical particles.

Polyoxyalkylenated silicone elastomers were in particular described in documents U.S. Pat. No. 5,236,986, U.S. Pat. No. 5,412,004, U.S. Pat. No. 5,837,793 and U.S. Pat. No. 5,811,487, the content of which is incorporated by way of reference.

As silicone elastomers comprising at least one oxyethylenated chain, use may in particular be made of those sold by the company Shin Etsu under the names:
KSG-21 (at 27% in terms of active material. INCI name: Dimethicone/PEG-10 Dimethicone vinyl dimethicone crosspolymer),
KSG-20 (at 100% in terms of active material. INCI name: PEG-10 Dimethicone Crosspolymer),
KSG-210 (at 256 in terms of active material. INCI name: Dimethicone/PEG-10/15 crosspolymer),
X-22-6146 (at 32% in terms of active material. INCI name: Dimethicone/PEG-10 Dimethicone vinyl dimethicone crosspolymer),
   or those sold by the company Dow Corning under the names:
DC9010 (at 11% in terms of active material. INCI name: PEG-12 dimethicone crosspolymer),
DC9011 (at 91% in terms of active material).

These products are generally in the form of oily gels containing the particles of silicone elastomer.

Use is preferably made of KSG-210 (INCI name: Dimethicone/PEG-10/15 crosspolymer), which is at 25% in terms of active material of silicone elastomer in silicone oil.

The emulsifying silicone elastomer may also be chosen from silicone elastomers comprising at least one glycerylated chain.

The silicone elastomer comprising at least one glycerylated chain can be obtained in particular by addition reaction and crosslinking of a diorganopolysiloxane containing at least one hydrogen linked to the silicon (A2) and of a polyglycerylated compound having ethylenically unsaturated groups (B2), in particular in the presence of a catalyst (C2), in particular a platinum catalyst.

In particular, the organopolysiloxane may be obtained by reaction of a polyglycerylated compound containing dimethylvinylsiloxy end groups and of methylhydrogenopolysiloxane containing trimethylsiloxy end groups, in the presence of a platinum catalyst.

Compound (A2) is the base compound for the formation of elastomeric organopolysiloxane, and the crosslinking takes place via an addition reaction of compound (A) with compound (B2) in the presence of catalyst (C2).

Compound (A2) is in particular an organopolysiloxane having at least two hydrogen atoms linked to distinct silicon atoms in each molecule. Compound (A2) may have a viscosity at 25° C. ranging from 1 to 50 000 centi-stokes, in particular so as to be readily miscible with compound (B2).

The organic groups linked to the silicon atoms of compound (A2) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, said organic group is chosen from methyl, phenyl and lauryl groups.

Compound (A2) may thus be chosen from methylhydrogenopolysiloxanes containing trimethylsiloxy end groups, dimethylsiloxane-methylhydrogenosiloxane copolymers containing trimethylsiloxy end groups, cyclic dimethylsiloxane-methylhydrogenosiloxane copolymers, and dimethylsiloxane-methylhydrogenosiloxane-laurylmethyl-siloxane copolymers containing trimethylsiloxy end groups.

Compound (B2) may be a polyglycerylated compound corresponding to formula (B′2) below:

CₘH₂ₘ₋₁—O-[Gly]ₙ-CₘH₂ₘ₋₁ (B′2)

in which m is an integer ranging from 2 to 6, n is an integer ranging from 2 to 200, preferably from 2 to 100, preferentially from 2 to 50, better still from 2 to 20, even better still from 2 to 10, and even better still from 2 to 5, and in particular n is equal to 3; and Gly denotes:

—CH₂—CH(OH)—CH₂—O— or —CH₂—CH(CH₂OH)—O—

Advantageously, the sum of the number of ethylenic groups per molecule of compound (B2) and of the number of hydrogen atoms linked to silicon atoms per molecule of compound (A2) is at least 4.

It is advantageous for compound (A2) to be added in an amount such that the molecular ratio of the total amount of hydrogen atoms linked to silicon atoms in compound (A2) to the total amount of all the ethylenically unsaturated groups in compound (B2) is within the range of from 1/1 to 20/1.

Compound (C2) is the crosslinking reaction catalyst, and is in particular chosen from chloroplatinic acid, chloroplatinic acid-olefin complexes, chloroplatinic acid-alkenylsiloxane complexes, chloroplatinic acid-diketone complexes, platinum black and platinum on a support.

The catalyst (C2) is preferably added in an amount of from 0.1 to 1000 parts by weight, better still from 1 to 100 parts by weight, as clean platinum metal, per 1000 parts by weight of the total amount of compounds (A2) and (B2).

The silicone elastomer comprising at least one glycerylated chain, used according to the invention, is generally in the form of a gel as a mixture with at least one hydrocarbon-based oil and/or one silicone oil. In these gels, the elastomer comprising at least one glycerylated chain is commonly in the form of non-spherical particles.

Such elastomers are in particular described in document WO-A-2004/024798.

As silicone elastomer comprising at least one glycerylated chain, use may be made of that sold by the company Shin Etsu under the name KSG-710 (containing 25% active material. INCI name: Dimethicone/Polyglycerin-3 Crosspolymer).

Preferably, the amount (in terms of active material) of emulsifying silicone elastomer(s) in the composition of the invention ranges from 0.1% to 20% by weight, better still from 0.5% to 16% by weight, even better still from 0.5% to 10% by weight, and even better still from 0.5% to 5% by weight, relative to the total weight of the composition.

The emulsifying silicone elastomer is generally introduced into the oily phase and is part of this oily phase.

### Semi-Crystalline Polymers

The composition according to the invention contains at least one semi-crystalline polymer, preferably derived from acrylic or methacrylic acid. For the purpose of the invention, the term “semi-crystalline polymer” is intended to mean polymers comprising a crystallizable portion, a crystallizable pendent chain or a crystallizable block in the backbone, and an amorphous portion in the backbone, and having a first-order reversible phase-change temperature, in particular of melting (solid-liquid transition). When the crystallizable portion is a block of the polymeric backbone, this crystallizable block is of chemical nature different from that of the amorphous blocks; in this case, the semi-crystalline polymer is a block polymer, for example of the diblock, triblock or multi-block type.

Advantageously, the semi-crystalline polymer(s) of the composition of the invention has (have) a number-average molecular mass Mn of greater than or equal to 2000, for example ranging from 2000 to 800 000, preferably from 3000 to 500 000, for example from 4000 to 150 000, and better still from 4000 to 99 000.

In the composition according to the invention, the semi-crystalline polymers are advantageously soluble in the oily phase to at least 1% by weight, at a temperature above their melting point. Besides the crystallizable chains or blocks, the polymer blocks are amorphous. For the purpose of the invention, the term “crystallizable chain or block” is intended to mean the chain or block which, if it were alone, would pass reversibly from the amorphous state to the crystalline state, depending on whether the temperature is above or below the melting point. For the purpose of the invention, a chain is a group of atoms, which is pendent or lateral relative to the polymer backbone. A block is a group of atoms belonging to the backbone, this group constituting one of the repeating units of the polymer.

Preferably, the polymer backbone of the semi-crystalline polymers is soluble in the oily phase.

Preferably, the semi-crystalline polymers used in the composition of the invention have a melting temperature (or melting point), Mp, of less than 70° C. (25° C.≦Mp<70° C.), this temperature being at least equal to the temperature of the keratin material that is to receive the composition according to the invention, especially the skin. The melting point may be measured especially by any known method, and in particular with a differential scanning calorimeter (DSC).

Preferably, the crystallizable blocks or chains of the semi-crystalline polymers represent at least 30% of the total weight of each polymer, and better still at least 40%. The semi-crystalline polymers containing crystallizable blocks used according to the invention are block or multiblock polymers. They may be obtained by polymerization of monomers containing reactive double bonds (or ethylenic bonds) or by polycondensation. When the polymers of the invention are polymers containing crystallizable side chains, they are advantageously in random or statistical form.

The semi-crystalline polymers of the invention are of synthetic origin. In addition, they comprise no polysaccharide backbone.

The semi-crystalline polymers that can be used in the invention are preferably chosen from polymers (homopolymers or copolymers) bearing at least one crystallizable side chain, and polymers (homopolymers or copolymers) bearing in the backbone at least one crystallizable block, such as those described in document U.S. Pat. No. 5,156,911. The crystallizable side chain(s) or block(s) is (are) hydrophobic.

According to a preferred embodiment of the invention, the semi-crystalline polymers are chosen in particular from homopolymers and copolymers resulting from the polymerization of at least one monomer with (a) crystallizable chain(s), said chain(s) being chosen from alkyl chains containing at least 11 carbon atoms and no more than 40 carbon atoms, and better still no more than 24 carbon atoms. They are in particular alkyl chains containing at least 12 carbon atoms, and they are preferably alkyl chains containing from 14 to 24 carbon atoms (C₁₄-C₂₄). They may be hydrocarbon-based alkyl chains (carbon and hydrogen atoms) or fluoro or perfluoroalkyl chains (carbon atoms, fluorine atoms and possibly hydrogen atoms). When they are fluoro or perfluoroalkyl chains, they contain at least 11 carbon atoms, at least 6 carbon atoms of which are fluorinated.

For the purpose of the invention, the term “alkyl” is intended to mean a saturated group (containing no unsaturation).

According to a particular embodiment of the invention, the semi-crystalline polymer is chosen from homopolymers obtained by polymerization of at least one monomer with a crystallizable chain, chosen from C₁₄-C₂₄ alkyl (meth)acrylates, C₁₁-C₁₅ perfluoroalkyl (meth)acrylates, C₁₄-C₂₄ N-alkyl (meth)acrylamides with or without fluorine atoms, vinyl esters containing C₁₄-C₂₄ alkyl or perfluoroalkyl chains, vinyl ethers containing C₁₄-C₂₄ alkyl or perfluoroalkyl chains, C₁₄-C₂₄ alpha-olefins, para-alkylstyrenes with a C₁₄-C₂₄ alkyl group, and from copolymers of these monomers, obtained by copolymerization of these monomers with a hydrophilic monomer, which is preferably other than methacrylic acid, for instance N-vinylpyrrolidone, hydroxyethyl acrylate, hydroxyethyl methacrylate or acrylic acid. Such copolymers may, for example, be copolymers of (C₁₄-C₂₄)alkyl acrylate, of (C₁₄-C₂₄)alkyl methacrylate, of (C₁₄-C₂₄)alkylacrylamide or of (C₁₄-C₂₄) alkylmethacrylamide with N-vinylpyrrolidone, hydroxyethyl acrylate, hydroxyethyl methacrylate or acrylic acid, or mixtures thereof.

Preferably, the semi-crystalline polymer is chosen from homopolymers obtained by polymerization of a monomer chosen from C₁₄-C₂₄ alkyl acrylates and C₁₄-C₂₄ alkyl methacrylates and from copolymers obtained by copolymerization of a monomer chosen from C₁₄-C₂₄ alkyl acrylates and C₁₄-C₂₄ alkyl methacrylates, with a hydrophilic monomer such as acrylic acid.

The semi-crystalline polymers of the composition of the invention may be non-crosslinked or partially crosslinked, provided that the degree of crosslinking does not hinder their dissolution or dispersion in the oily phase by heating above their melting point. It may then be a case of chemical crosslinking, by reaction with a multifunctional monomer during the polymerization. It may also be a case of physical crosslinking, which may then be due either to the establishment of bonds of hydrogen or dipolar type between the groups borne by the polymer, for instance dipolar interactions between carboxylate ionomers, these interactions being in small amount and borne by the polymer backbone; or to a phase separation between the crystallizable blocks and the amorphous blocks borne by the polymer.

Preferably, the semi-crystalline polymers of the composition according to the invention are non-crosslinked.

According to a particular embodiment of the invention, the semi-crystalline polymer is a homopolymer resulting from the polymerization of a monomer with a crystallizable chain chosen from C₁₄-C₂₄ alkyl acrylates and C₁₄-C₂₄ alkyl methacrylates. Mention may in particular be made of those sold under the names Intelimer® by the company Landec, described in the brochure “Intelimer® polymers”, Landec IP22. These polymers are in solid form at ambient temperature. They bear crystallizable side chains and correspond to saturated C₁₄-C₂₄ alkyl acrylate or methacrylate homopolymers. Mention may more particularly be made of the stearyl acrylate homopolymer (Intelimer IPA-13.1) (INCI name: Poly C10-30 alkyl acrylate) and the behenyl acrylate homopolymer (Intelimer IPA-13.6 (INCI name: Poly C10-30 alkyl acrylate).

According to another particular embodiment of the invention, the semi-crystalline polymer is a copolymer of C₁₄-C₂₄ alkyl acrylates or of C₁₄-C₂₄ alkyl methacrylates with acrylic acid. As copolymers of this type, mention may be made of the copolymers obtained by copolymerization of behenyl acrylate and of acrylic acid, and the copolymers obtained by copolymerization of stearyl acrylate and of acrylic acid.

According to a preferred embodiment of the invention, the semi-crystalline polymer is a homopolymer, and it is chosen from the stearyl acrylate homopolymer (Intelimer IPA-13.1) (INCI name: Poly C10-30 alkyl acrylate) and the behenyl acrylate homopolymer (Intelimer IPA-13.6) (INCI name: Poly C10-30 alkyl acrylate), and mixtures thereof.

The amount of semi-crystalline polymer(s) in the composition of the invention may range, for example, from 0.1% to 5% by weight, preferably from 0.5% to 5% by weight, and better still from 1% to 4% by weight, relative to the total weight of the composition.

### 2-Acrylamido-2-methylpropanesulphonic acid polymers

According to a particular embodiment of the invention, the composition may also contain one or more 2-acrylamido-2-methylpropanesulphonic acid polymers. The addition of such a polymer has the advantage of reinforcing the stability of the emulsions even in the absence of electrolyte.

In the present application, the expression “polymer comprising 2-acrylamido-2-methylpropanesulphonic acid units” (AMPS) is intended to mean both homopolymers and copolymers, and both crosslinked polymers and non-crosslinked polymers.

They are water-soluble or water-dispersible or water-swellable polymers.

Preferably, the AMPS polymers used in accordance with the invention may be partially or completely neutralized with an inorganic base (sodium hydroxide, potassium hydroxide, aqueous ammonia) or an organic base such as mono-, di- or triethanolamine, an aminomethylpropanediol, N-methylglucamine or basic amino acids such as arginine and lysine, and mixtures of these compounds. They are generally neutralized. In the present invention, the term “neutralized” is intended to mean polymers that have been completely or almost completely neutralized, i.e. at least 90% neutralized.

The AMPS polymers used in the composition of the invention generally have a number-average molecular weight ranging from 1000 to 20 000 000 g/mol, preferably ranging from 20 000 to 5 000 000, and even more preferably from 100 000 to 1 500 000 g/mol.

When the polymers are crosslinked, the crosslinking agents may be chosen from compounds with an olefinic polyunsaturation commonly used for crosslinking polymers obtained by radical polymerization. As crosslinking agents, mention may, for example, be made of divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric acid derivatives and/or vinylphosphonic acid derivatives, or mixtures of these compounds. According to a preferred embodiment of the invention, the crosslinking agent is chosen from methylenebisacrylamide, allyl methacrylate and tri-methylolpropane triacrylate (TMPTA). The degree of crosslinking generally ranges from 0.01 to 10 mol %, and more particularly from 0.2 to 2 mol %, relative to the polymer.

The AMPS homopolymers preferred for use in the composition of the invention are crosslinked and neutralized, and they can be obtained according to the preparation process comprising the following steps:

(a) 2-acrylamido-2-methylpropanesulphonic acid in free form is dispersed or dissolved in a solution of tert-butanol or of water and tert-butanol;
(b) the solution or the dispersion of monomer obtained in (a) is neutralized with one or more inorganic or organic bases, preferably aqueous ammonia NH₃, in an amount that makes it possible to obtain a degree of neutralization of the sulphonic acid functional groups of the polymer ranging from 90% to 100%;
(c) the crosslinking monomer(s) is (are) added to the solution or dispersion obtained in (b);
(d) a conventional radical polymerization is carried out in the presence of free radical initiators at a temperature ranging from 10 to 150° C.; the polymer precipitating from the tert-butanol-based solution or dispersion.

The AMPS homopolymers that are more particularly preferred comprise, distributed randomly, units of general formula (I) below:

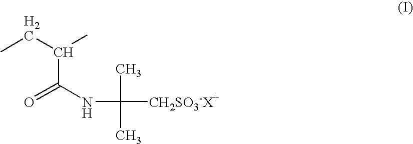

in which X⁺ denotes a proton, an alkali metal cation, an alkaline earth metal cation or the ammonium ion, it being possible for no more than 10 mol % of the X⁺ cations to be H⁺ protons;
and crosslinking units originating from at least one monomer having at least two olefinic double bonds.

The homopolymers used according to the invention and that are more particularly preferred comprise for example from 90% to 99.9% by weight, and preferably from 98% to 99.5% by weight of units of formula (I), and from 0.01% to 10% by weight, preferably from 0.2% to 2% by weight of crosslinking units, the proportions by weight being defined relative to the total weight of the polymer.

As homopolymer of this type, mention may in particular be made of the crosslinked and neutralized 2-acrylamido-2-methylpropanesulphonic acid homopolymer sold by the company Clariant under the trade name “Hostacerin AMPS” (INCI name: Ammonium Polyacryldimethyltauramide).

The 2-acrylamido-2-methylpropanesulphonic acid (AMPS) copolymers that can be used in the composition of the invention may be chosen in particular from:

1) crosslinked anionic copolymers of acrylamide or methacrylamide and of 2-acrylamido-2-methylpropanesulphonic acid, in particular those which are in the form of a W/O emulsion, such as those sold under the name Sepigel 305 by the company Seppic (INCI name: Polyacrylamide/C13-14 Isoparaffin/Laureth-7), or under the name Simulgel 600 by the company Seppic (INCI name: Acrylamide/Sodium acryloyldimethyltaurate copolymer/isohexadecane/Polysorbate 80),
2) copolymers of (meth)acrylic acid or of (meth)acrylate and of 2-acrylamido-2-methylpropane-sulphonic acid, in particular those which are in the form of a W/O emulsion, such as those sold under the name Simulgel NS by the company Seppic (copolymer of sodium acrylamido-2-methylpropanesulphonate/hydroxylethyl acrylate in an inverse emulsion at 40% in polysorbate 60 and squalene) (INCI name: Hydroxyethyl acrylate/sodium acryloyldimethyltaurate copolymer/squalene/polysorbate 60) or those sold under the name Simulgel EG by the company Seppic (copolymer of acrylic acid/acrylamido-2-methylpropanesulphonic acid in the form of a sodium salt in an inverse emulsion at 45% in isohexadecane/water) (INCI name: sodium acrylate/sodium acryloyldimethyltaurate copolymer/isohexadecane/Polysorbate 80),
3) copolymers of 2-acrylamido-2-methylpropanesulphonic acid and of vinylpyrrolidone or of vinylformamide, such as the products sold under the names Aristoflex AVC by the company Clariant,
4) copolymers of 2-acrylamido-2-methylpropanesulphonic acid containing a hydrophobic unit, in particular the copolymers comprising a 2-acrylamido-2-methylpropane-sulphonic acid unit of formula (I) as defined above, and at least one hydrophobic unit of formula (II)

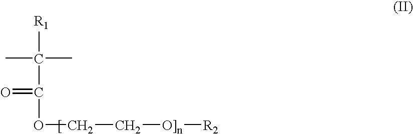

in which n denotes a number of moles which is an integer ranging from 3 to 100, preferably from 3 to 50, and more preferably from 7 to 25, R₁ is hydrogen or a methyl radical, and R₂ denotes a linear or branched alkyl radical containing from 6 to 30 carbon atoms, preferably from 10 to 22 carbon atoms, and better still from 14 to 22 carbon atoms.

In these copolymers, the AMPS unit of formula (I) represents in general from 80 to 99 mol %, and preferably from 85 to 99 mol %, and the unit of formula (II) represents in general from 1 to 20 mol %, and preferably from 1 to 15 mol %.

As AMPS copolymers containing a hydrophobic unit, mention may in particular be made of the copolymer of AMPS and of ethoxylated C₁₂-C₁₄ alkyl methacrylate (non-crosslinked copolymer obtained from Genapol LA-070 and from AMPS) (INCI name: Ammonium Acryloyldimethyl-taurate/Laureth-7 Methacrylate Copolymer) sold under the name Aristoflex LNC by the company Clariant, the copolymer of AMPS and of ethoxylated stearyl methacrylate (25 EO) (copolymer crosslinked with trimethylolpropane triacrylate, obtained from Genapol T-250 and from AMPS) (INCI name: Ammonium Acryloyldimethyltaurate/Steareth 25 Methacrylate Crosspolymer) sold under the name Aristoflex HMS by the company Clariant, and the copolymer of AMPS and of ethoxylated C₁₆-C₁₈ alkyl methacrylate (INCI name: Ammonium Acryloyldimethyltaurate/Steareth-8 Methacrylate Copolymer), a non-crosslinked copolymer obtained from Genapol T-080 and from AMPS, sold under the name Aristoflex SNC by the company Clariant.

According to a preferred embodiment of the invention, the polymer is an AMPS homopolymer, and in particular Hostacerin AMPS.

The amount of 2-acrylamido-2-methylpropanesulphonic acid polymer(s) in the composition of the invention may range, for example, in terms of active material, from 0.1% to 5% by weight, preferably from 0.2% to 5% by weight, better still from 0.2% to 3% by weight, relative to the total weight of the composition.

### Oily Phase

The oily phase consists of the oils and of all the other fatty substances and lipophilic constituents that may be present in the composition of the invention, including the silicone elastomer and the lipophilic polymer. All the cosmetically acceptable oils may be used.

The term “oil” is intended to mean a fatty substance that is liquid at ambient temperature (25° C.).

As oils that can be used in the composition of the invention, mention may, for example, be made of:
hydrocarbon-based oils of animal (fish) origin, such as perhydrosqualene and squalene;
hydrocarbon-based oils of plant origin, such as liquid triglycerides of fatty acids containing from 4 to 10 carbon atoms, for instance heptanoic or octanoic acid triglycerides, or alternatively, for example, sunflower oil, maize oil, soybean oil, marrow oil, grapeseed oil, sesame oil, hazelnut oil, apricot oil, macadamia oil, arara oil, castor oil, avocado oil, caprylic/capric acid triglycerides, such as those sold by the company Stéarineries Dubois or those sold under the names Miglyol 810, 812 and 818 by the company Dynamit Nobel, jojoba oil and shea butter oil;
synthetic esters and ethers, in particular fatty acid esters and ethers, such as the oils of formulae R¹COOR² and R¹OR² in which R¹ represents the residue of a fatty acid or of a fatty alcohol containing from 8 to 29 carbon atoms and R² represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate, isostearyl isostearate or isononyl isononanoate; hydroxylated esters, for instance isostearyl lactate, octyl hydroxystearate, octyldodecyl hydroxylstearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate (Prisorine 3631);
linear or branched hydrocarbons of mineral or synthetic origin, such as volatile or non-volatile liquid paraffins, and derivatives thereof, petroleum jelly, polydecenes, isohexadecane, isododecane, and hydrogenated polyisobutene such as Parleam oil;
silicone oils, for instance volatile or non-volatile polymethylsiloxanes (PDMSs) containing a linear or cyclic silicone chain, which are liquid or pasty at ambient temperature, in particular cyclopolydimethylsiloxanes (cyclomethicones) such as cyclohexasiloxane and cyclopentasiloxane; polydimethylsiloxanes (or dimethicones) comprising alkyl, alkoxy or phenyl groups, which groups are pendent or at the end of a silicone chain and contain from 2 to 24 carbon atoms; phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethyl siloxysilicates and polymethylphenylsiloxanes;
fatty alcohols containing from 8 to 26 carbon atoms, such as cetyl alcohol, stearyl alcohol and the mixture thereof (cetearyl alcohol);
partially hydrocarbon-based and/or silicone-based fluoro oils, such as those described in document JP-A-2-295912;
and mixtures thereof.

The term “hydrocarbon-based oil” used above is intended to mean any oil comprising predominantly carbon and hydrogen atoms and, optionally, ester, ether, fluoro, carboxylic acid and/or alcohol groups.

According to a preferred embodiment of the invention, the oily phase of the composition contains at least 50% by weight of non-volatile oils relative to the total weight of the oily phase, and the volatile oils therefore represent no more than 50% of the total weight of the oily phase. The term “volatile oil” is intended to mean an oil (or nonaqueous medium) capable of evaporating on contact with the skin in less than one hour, at ambient temperature and atmospheric pressure. The volatile oil is an oil that is liquid at ambient temperature, and has a non-zero vapour pressure, at ambient temperature and atmospheric pressure, in particular has a vapour pressure ranging from 0.13 Pa to 40 000 Pa (10⁻³ to 300 mmHg), and preferably ranging from 1.3 Pa to 13 000 Pa (0.01 to 100 mmHg), and preferentially ranging from 1.3 Pa to 1300 Pa (0.01 to 10 mmHg).

According to a preferred embodiment of the invention, the composition contains at least one oil chosen from hydrogenated polyisobutene (INCI name: Hydrogenated Polyisobutene or C13-16 Isoparaffin) and fatty acid esters such as, in particular, isononyl isononanoate, isopropyl myristate, 2-ethylhexyl palmitate, 2-octyl-dodecyl stearate, 2-octyldodecyl erucate, isostearyl isostearate or isononyl isononanoate, and mixtures thereof.

In addition to the oils indicated above, the composition of the invention may contain other fatty substances in the oily phase, such as fatty acids containing from 8 to 30 carbon atoms, for instance stearic acid; silicone resins such as trifluoromethyl(C₁-C₄)alkyl dimethicone and trifluoropropyl dimethicone; silicone gums (dimethiconol), non-emulsifying silicone elastomers, such as the products sold under the names “KSG 6” or “KSG 16” by the company Shin-Etsu, under the names “Trefil”, “BY29” or “EPSX” by the company Dow Corning, or under the names “Gransil” by the company Grant Industries; waxes, for example mineral waxes, waxes of animal origin, for instance beeswax, waxes of plant origin, hydrogenated oils that are solid at 25° C., fatty esters and glycerides that are solid at 25° C., synthetic waxes such as polymethylene wax or silicone waxes; and mixtures thereof. These various constituents are non-volatile and are part of the oily phase.

The oily phase (comprising all the lipophilic constituents) may be present in the composition according to the invention in an amount ranging, for example, from 5% to 60%, preferably from 10% to 50% by weight, better still from 10% to 40% by weight, and better still from 15% to 30% by weight, relative to the total weight of the composition. According to a particularly preferred embodiment of the invention, the oily phase represents no more than 40% of the total weight of the composition.

### Aqueous Phase

The amount of aqueous phase may range for example from 40% to 95% by weight, preferably from 50% to 90% by weight, better still from 60% to 90% by weight, and even better still from 70% to 85% by weight, relative to the total weight of the composition. According to a particularly preferred embodiment of the invention, the aqueous phase represents at least 60% of the total weight of the composition.

The aqueous phase comprises water and any hydrophilic adjuvants (solvents, active agents and additives). The water preferably represents at least 30%, and better still at least 40% of the total weight of the composition, and for example from 30% to 95%, preferably from 40% to 90% by weight, and better still from 50% to 80% by weight, relative to the total weight of the composition.

As hydrophilic solvents that may be used in the composition of the invention, mention may in particular be made of monoalcohols containing 2 to 8 carbon atoms, such as ethanol or isopropanol, and mixtures thereof.

### Adjuvants

In a known manner, the composition of the invention may also contain adjuvants including those that are common in the cosmetics field, such as polyols, active agents, preserving agents, antioxidants, electrolytes, complexing agents, fragrances, fillers, bactericides, odour absorbers, dyestuffs (dyes and pigments) and lipid vesicles. The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

These adjuvants and also their concentrations should be such that they do not modify the properties sought for the composition of the invention.

As polyols, mention may in particular be made of glycerol, glycols such as pentylene glycol, propylene glycol, butylene glycol or isoprene glycol and polyethylene glycols such as PEG-8; sorbitol; sugars such as glucose, fructose, maltose, lactose or sucrose; and mixtures thereof.

As examples of active agents that can be used in the composition according to the invention, mention may, for example, be made of (N-2-hydroxyethylpiperazine-N-2-ethane)sulphonic acid (HEPES); hyaluronic acid; lanolin; urea, mixtures containing urea, such as NMF (natural moisturizing factor), and urea derivatives such as N-(2-hydroxyethyl)urea (Hydrovance from the company National Starch); 2-oxothiazolidine-4-carboxylic acid (procysteine); α-hydroxy acids, in particular acids derived from fruits, such as glycolic acid, lactic acid, malic acid, citric acid, tartaric acid or mandelic acid, derivatives thereof and mixtures thereof; β-hydroxy acids, for instance salicylic acid and its derivatives such as 5-n-octanoylsalicylic acid or 5-n-dodecanoylsalicylic acid; α-keto acids, for instance ascorbic acid or vitamin C and its derivatives such as its salts, for instance sodium ascorbate, magnesium ascorbylphosphate or sodium ascorbylphosphate; its esters such as ascorbyl acetate, ascorbyl palmitate and ascorbyl propionate, or its sugars such as glycosylated ascorbic acid, and mixtures thereof; β-keto acids; retinoids such as retinol (vitamin A) and its esters, retinal, retinoic acid and its derivatives, and also the retinoids described in documents FR-A-2,570,377, EP-A-199 636, EP-A-325 540, EP-A-402 072, EP-A-325 540 and EP-A-402 072; carotenoids such as lycopene; ceramides; sapogenins and plant extracts containing them, in particular extracts of wild yam; resveratrol; pseudodipeptides such as {2-[acetyl-(3-trifluoromethylphenyl)amino]-3-methyl-butyrylamino}acetic acid; vitamins such as, for example, in addition to the vitamins A and C indicated above, vitamin E (tocopherol), vitamin B3 (or vitamin PP or niacinamide), vitamin B5 (panthenol in its various forms: D-panthenol, DL-panthenol), vitamin D, vitamin F (mixture of essential fatty acids), the derivatives, precursors and analogues of these vitamins; soybean extracts, in particular soybean protein hydrolysates or soybean extracts rich in isoflavones; trace elements such as copper, zinc, selenium, iron, magnesium or manganese; algal extracts such as the products sold under the name Stimoderm by the company CLR; plankton extracts such as the plankton in an aqueous dispersion (INCI name: Vitreoscilla Ferment) sold under the name Mexoryl SAH by the company Chimex; enzymes; coenzymes such as ubiquinone or coenzyme Q10 which belong to the alkylenated-chain benzoquinone family, coenzyme R which is biotin (or vitamin H); yeast extracts such as the S. cerevisiae extract sold under the name Cytovitin LS 9388 by the Laboratoires Sériobiologiques; adenosine; plant extracts such as liquorice extracts; calmatives such as bisabolol and calming plant extracts such as extracts of rose (Rosa gallica) and extracts of mint (Mentha piperita); C-glycoside derivatives such as C-β-D-xylo-pyranoside-n-propan-2-one, for example in the form of a solution containing 30% active material in a water/propylene glycol mixture (60/40% by weight), such as the product manufactured by the company Chimex under the trade name Mexoryl SBB®; and any active agent suitable for the final purpose of the invention, and mixtures thereof.

The composition may contain one or more fillers. Fillers that the composition of the invention may contain include, for example, mineral particles such as clays, silicas, metal oxides such as titanium dioxide or zinc oxide, or mica, and/or organic fillers such as polyamide (Nylon) particles and in particular those sold under the name Orgasol by the company Atochem; polyethylene powders; microspheres based on acrylic copolymers, such as those of ethylene glycol dimethacrylate/lauryl methacrylate copolymer sold by the company Dow Corning under the name Polytrap; the poly(methyl methacrylate) microspheres sold under the name Microsphere M-100 by the company Matsumoto or under the name Covabead LH85 by the company Wackherr; ethylene acrylate copolymer powders, such as those sold under the name Flobeads by the company Sumitomo Seika Chemicals; expanded powders such as hollow microspheres, and in particular the microspheres formed from a terpolymer of vinylidene chloride, acrylonitrile and methacrylate and sold under the name Expancel by the company Kemanord Plast; powders of natural organic materials, such as starch powders, in particular crosslinked or non-crosslinked maize, wheat or rice starch powders, such as the powders of starch crosslinked with octenylsuccinate anhydride, sold under the name Dry-Flo by the company National Starch; silicone resin microbeads such as those sold under the name Tospearl by the company Toshiba Silicone, in particular Tospearl 240; and mixtures thereof. The amount of filler(s) may range, for example, from 0.05% to 10% by weight, and better still from 0.1% to 5% by weight, relative to the total weight of the composition.

The composition according to the invention may advantageously be in the form of a more or less fluid cream, i.e. a product which has a certain consistency but is flexible, as opposed to a solid product such as a stick. Thus, this composition may have a viscosity at 25° C. ranging from approximately 10 to 200 poises (1 to 20 Pa·s), preferably from approximately 15 to 150 poises (1.5 to 15 Pa·s), and better still from 20 to 100 poises (2 to 10 Pa·s), this viscosity being measured with a Rhéomat 180 device at 25° C. and with the appropriate spindles.

The cosmetic composition according to the invention finds its application in a large number of cosmetic treatments for keratin materials, and more especially for the skin, in particular in skincare, for nourishing the skin, preventing transepidermal water loss and protecting the skin.

Thus, a subject of the present invention is the cosmetic use of the composition as defined above, for skincare, in particular for nourishing the skin, preventing transepidermal water loss and/or protecting the skin.

The composition is particularly suitable for the treatment of dry skin.

A subject of the present invention is also a cosmetic process for treating dry skin, comprising the application to the skin of the composition as defined above.

The following examples of compositions according to the invention are given by way of illustration and are not limiting in nature. The compounds are indicated by chemical name or by INCI name. The amounts thereof are given as % by weight of starting material, unless otherwise mentioned.

### EXAMPLES 1 AND 2 ACCORDING TO THE INVENTION AND COMPARATIVE EXAMPLE 1

| | Example 1 | Example 2 | |
|---|---|---|---|
| | according | according | |
| | to the | to the | Comparative |
| Composition | invention | invention | example 1 |
| Aqueous phase | | | |
| Glycerol | 7 | 7 | 7 |
| Hostacerin AMPS | 1 | 1 | 1 |
| Preserving agent | 0.4 | 0.4 | 0.4 |
| Water | qs 100% | qs 100% | qs 100% |
| Oily phase | | | |
| KSG210 at 25% in | 6.6 | 6.6 | 6.6 |
| terms of active | | | |
| material (i.e. 1.65% | | | |
| of A.M.) | | | |
| Behenyl acrylate | 2 | 2 | 0 |
| homopolymer | | | |
| (Intelimer IPA-13.6) | | | |
| Cyclohexamethicone | 5 | 3 | 7 |
| Hydrogenated | 5 | — | 5 |
| polyisobutene | | | |
| (Parleam oil) | | | |
| Isohexadecane | 5 | 5 | 5 |
| Result in terms of | Stable | Stable | Instable |
| stability (2 months | | | |
| at 45° C.) | | | |

Protocol: Each phase was homogenized and heated to 65° C. The aqueous phase was then poured slowly into the oily phase.

The compositions of the invention are in the form of a cream and are stable, unlike the composition of the comparative example containing no semi-crystalline polymer.

### EXAMPLE 3 ACCORDING TO THE INVENTION

| Aqueous phase | |
|---|---|
| Preserving agent | 0.4% |
| AMPS polymer (Hostacerin AMPS) | 1% |
| Water | qs 100% |
| Oily phase | |
| KSG 210 (at 25% in terms of active material) | 3% |
| (i.e. 0.75% of active material) | |
| Stearyl acrylate homopolymer (Intelimer IPA- | 2% |
| 13.1) | |
| Hydrogenated polyisobutene (Parleam oil) | 10% |
| Isohexadecane | 5% |

The protocol is identical to that indicated for the previous examples.

A thick, homogeneous, smooth cream with a viscosity of 80 poises (i.e. 8 Pa·s) (measurement carried out at 25° C., 10 minutes after preparation, with a Rhéomat 180, spindle 4) was obtained. This cream was fresh on application while at the same time being nutritive. Under the microscope, the emulsion was even with sharp edges. The composition remained stable after the centrifugation test.

### COMPARATIVE EXAMPLE 2

| Aqueous phase | |
|---|---|
| Preserving agent | 0.4% |
| AMPS polymer (Hostacerin AMPS) | 1% |
| Water | qs 100% |
| Oily phase | |
| KSG 210 (at 25% in terms of active material) | 3% |
| (i.e. 0.75% of active material) | |
| Hydrogenated polyisobutene (Parleam oil) | 10% |
| Isohexadecane | 5% |

The protocol is identical to that indicated for the previous examples.

A product more fluid than that of example 3 according to the invention was obtained, its viscosity being 14 poises (i.e. 1.4 Pa·s) (measurement carried out at 25° C., 10 minutes after preparation, with a Rhéomat 180, spindle 3). In addition, the product was less smooth and more heterogeneous in appearance than that of example 3 according to the invention. Furthermore, under the microscope, the emulsion was less even with loose edges and areas of release. In addition, this emulsion proved to be unstable in the centrifugation test since it experienced phase separation. This comparative example shows that, in the absence of semi-crystalline polymer, the composition is no longer a cream and that it is less stable.

### EXAMPLE4 ACCORDING TO THE INVENTION: MOISTURIZING DAY CREAM

| Aqueous phase | |
|---|---|
| Preserving agent | 0.4% |
| Glycerol | 5% |
| AMPS polymer (Hostacerin AMPS) | 1% |
| Water | qs 100% |
| Oily phase | |
| KSG 210 (at 25% in terms of active material) | 4% |
| (i.e. 1% of active material) | |
| Stearyl acrylate homopolymer (Intelimer IPA- | 1% |
| 13.1) | |
| Isohexadecane | 5% |
| Cyclohexamethicone | 3% |
| Hydrogenated polyisobutene (Parleam oil) | 7% |

The protocol is identical to that indicated for the previous examples.

Quite a thick cream that melts on the skin was obtained; it was fresh and very pleasant to use. After application of this cream, the skin was soft, fresh and well-moisturized.

### EXAMPLE 5 ACCORDING TO THE INVENTION: NIGHT CREAM

| Aqueous phase | |
|---|---|
| Preserving agent | 0.4% |
| Glycerol | 5% |
| AMPS polymer (Hostacerin AMPS) | 1% |
| Water | qs 100% |
| Oily phase | |
| KSG 710 (at 25% in terms of active material) | 8% |
| (i.e. 2% of active material) | |
| Stearyl acrylate homopolymer (Intelimer IPA- | 2% |
| 13.1) | |
| Isohexadecane | 5% |
| Hydrogenated polyisobutene (Parleam oil) | 10% |

The protocol is identical to that indicated for the previous examples.

An emulsion which was fine, fresh and yet nutritive on application, which spread readily and which was rapidly absorbed into the skin, which it left soothed, smoothed and non-greasy, was obtained.

### EXAMPLE 6 ACCORDING TO THE INVENTION AND COMPARATIVE EXAMPLE 3

| | Example 6 | |
|---|---|---|
| | according | |
| | to the | Comparative |
| Composition | invention | example 3 |
| Aqueous phase | | |
| Glycerol | 5 | 5 |
| Magnesium sulphate | 0.7 | 0.7 |
| Preserving agent | 0.4 | 0.4 |
| Water | qs 100% | qs 100% |
| Oily phase | | |
| KSG210 at 25% in terms of | 16 | 16 |
| active material | | |
| (i.e. 4% of A.M.) | | |
| Behenyl acrylate homopolymer | 1 | 0 |
| (Intelimer IPA-13.6) | | |
| Jojoba oil | 5 | 5 |
| Polymethylene wax | 2 | 2 |
| Cyclohexamethicone | 5 | 5 |
| Hydrogenated polyisobutene | 10 | 10 |
| (Parleam oil) | | |
| Appearance under the | Stable | Areas of oil |
| microscope after 5 days | | release |

The preparation process was the same as for the other examples. Example 6 constitutes a cream which is suitable for the care of very dry skin. Comparative example 3 is more fluid and is less stable, despite the presence of oily-phase thickener (wax) and the presence of salt in the aqueous phase (magnesium sulphate).

### EXAMPLE 7 ACCORDING TO THE INVENTION: MOISTURIZING CREAM

| | |
|---|---|
| Aqueous phase | |
| Preserving agent | 0.4% |
| Glycerol | 7% |
| Magnesium sulphate | 0.7% |
| Water | qs 100% |
| Oily phase | |
| KSG 710 (at 25% in terms of active material) | 16% |
| (i.e. 4% of active material) | |
| Stearyl acrylate homopolymer (Intelimer IPA- | 1% |
| 13.1) | |
| Hydrogenated polyisobutene (Parleam oil) | 15% |
| Polymethylene wax | 1% |
| Cyclohexasiloxane | 5% |
| Fillers | |
| Maize starch esterified with octenyl succinic | 3% |
| anhydride (Dry Flo) | |

A smooth, rich, comfortable cream which leaves the skin matt, non-greasy and moisturized is obtained.

The above written description of the invention provides a manner and process of making and using it such that any person skilled in this art is enabled to make and use the same, this enablement being provided in particular for the subject matter of the appended claims, including a composition for topical application in the form of a water-in-oil emulsion comprising an aqueous phase dispersed in an oily phase, and containing at least one emulsifying silicone elastomer and at least one semi-crystalline polymer.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition in the form of a water-in-oil emulsion comprising an aqueous phase dispersed in an oily phase, at least one emulsifying silicone elastomer, and at least one semi-crystalline polymer.

2. The composition according to claim 1, wherein the emulsifying silicone elastomer is chosen from silicone elastomers comprising at least one oxyalkylenated chain and/or one glycerylated chain.

3. The composition according to claim 2, comprising a silicone elastomer comprising at least one oxyalkylenated chain obtained by addition reaction and crosslinking of diorganosiloxane comprising at least one hydrogen linked to the silicon and a polyoxyalkylene having at least two ethylenically unsaturated groups, in the presence of a catalyst.

4. The composition according to claim 1, wherein the emulsifying silicone elastomer is a silicone elastomer comprising at least one oxyethylenated chain.

5. The composition according to claim 2, comprising a silicone elastomer comprising at least one glycerylated chain obtained by addition reaction and crosslinking of diorganosiloxane comprising at least one hydrogen linked to the silicon and of polyglycerylated compounds having ethylenically unsaturated groups, in the presence of a catalyst.

6. The composition according to claim 1, wherein the amount of emulsifying silicone elastomer(s) ranges from 0.1% to 20% by weight relative to the total weight of the composition.

7. The composition according to claim 1, wherein the semi-crystalline polymer is chosen from homopolymers and copolymers resulting from the polymerization of at least one monomer comprising (a) crystallizable chain(s), said chain(s) being chosen from alkyl chains containing at least 11 carbon atoms and no more than 40 carbon atoms.

8. The composition according to claim 1, wherein the semi-crystalline polymer is chosen from homopolymers obtained by polymerization of a monomer chosen from C₁₄-C₂₄ alkyl acrylates and C₁₄-C₂₄ alkyl methacrylates and from copolymers obtained by copolymerization of a monomer chosen from C₁₄-C₂₄ alkyl acrylates and C₁₄-C₂₄ alkyl methacrylates, with a hydrophilic monomer.

9. The composition according to claim 1, wherein the semi-crystalline polymer is chosen from stearyl acrylate homopolymer and behenyl acrylate homopolymer, and mixtures thereof.

10. The composition according to claim 1, wherein the amount of semi-crystalline polymer(s) ranges from 0.1% to 5% by weight relative to the total weight of the composition.

11. The composition according to claim 1, further comprising one or more 2-acrylamido-2-methylpropanesulphonic acid polymers.

12. The composition according to claim 11, comprising a 2-acrylamido-2-methylpropanesulphonic acid polymer that is a homopolymer comprising, distributed randomly, units of general formula (I) below: 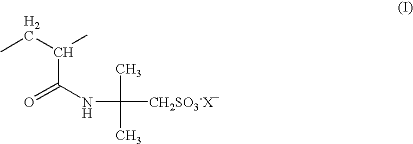
in which X⁺ denotes a proton, an alkali metal cation, an alkaline earth metal cation or an ammonium ion, it being possible for no more than 10 mol % of the X⁺ cations to be H⁺ protons;
and crosslinking units originating from at least one monomer having at least two olefinic double bonds.

13. The composition according to claim 11, comprising a 2-acrylamido-2-methylpropanesulphonic acid polymer that is a copolymer chosen from:
crosslinked anionic copolymers of acrylamide or methacrylamide and of 2-acrylamido-2-methylpropanesulphonic acid,
copolymers of (meth)acrylic acid or of (meth)acrylate and of 2-acrylamido-2-methylpropanesulphonic acid,
copolymers of 2-acrylamido-2-methylpropanesulphonic acid and of vinylpyrrolidone or of vinylformamide,
copolymers comprising a 2-acrylamido-2-methylpropanesulphonic acid unit of formula (I), and at least one hydrophobic unit of formula (II): 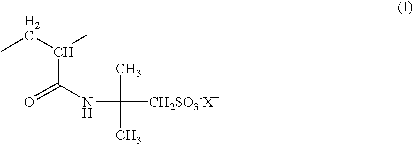
in which X⁺ denotes a proton, an alkali metal cation, an alkaline earth metal cation or an ammonium ion, it being possible for no more than 10 mol % of the X⁺ cations to be H⁺ protons; 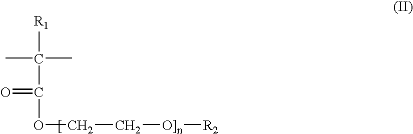
in which n denotes an integer ranging from 3 to 100, R₁, is hydrogen or a methyl radical, and R₂ denotes a linear or branched alkyl radical containing from 6 to 30 carbon atoms.

14. The composition according to claim 11, wherein the amount of 2-acrylamido-2-methylpropanesulphonic acid polymer(s) ranges from 0.1% to 5% by weight relative to the total weight of the composition.

15. The composition according to claim 1, wherein the oily phase comprises no more than 50% of volatile oils relative to the total weight of the oily phase.

16. The composition according to claim 1, comprising at least one oil chosen from hydrogenated polyisobutene and fatty acid esters, and mixtures thereof.

17. The composition according to claim 1, wherein the aqueous phase is present in an amount ranging from 40% to 95% relative to the total weight of the composition.

18. The composition according to claim 1, comprising at least 30% of water by weight relative to the total weight of the composition.

19. A method, comprising contacting the skin with the composition of claim 1.

20. The method according to claim 19, wherein said method is a method for nourishing the skin, preventing transepidermal water loss and/or protecting the skin.
